(19) United States
(12) Patent Application Publication
(10) Pub. No.: US 2011/0042258 A1
(43) Pub. Date: Feb. 24, 2011

(54) Funnel-Like Urinary Device

(75) Inventor: Marco Nett, Zurich (CH)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignees: Andreas B. Mattle, Bassersdorf (CH); Josef K. Stocker, Kaltbrunn (CH); Roger Meier, Schanis (CH)

(21) Appl. No.: 12/933,036

(22) PCT Filed: Mar. 18, 2008
(86) PCT No.: PCT/CH2008/000115

§ 371 Date: Nov. 11, 2010

(51) Int. Cl. A61F 5/455; A61B 19/02; B32B 38/00

(52) U.S. Cl. 206/494; 604/347; 156/227

(57) ABSTRACT

A funnel-like urinary device (1) comprises a funnel-like basic body (2) with mirror symmetry formed from a first wall (3) and a second wall (4), said first wall (3) and said second wall (4), if pulled apart, form a large opening (5) at a first end and a small opening (6) at a second end, the large opening (5) being designed such as to fit a woman's body in the vaginal region such that upon use it is sealed by the labia, and a second end defining a smaller opening (6) for discharging urine upon use and two holding means (7, 8) symmetrically positioned at the outside of the first wall (3) and the second wall (4), said holding means (7, 8) enabling the user to press the rim of the large opening (5) to the user's body in the vaginal region and to slightly open the large opening (5) such that its outside is in sealing contact with the labia.

### TECHNICAL FIELD

The present invention relates to a funnel-like urinary device allowing women to urinate in an up-right position, preferably a disposable device.

### BACKGROUND ART

A number of urinary devices for women are known in the art. Some of them are made of a liquid-resistant material, e.g. plastics, as disclosed in US 2007/0169253 A1. Others consist of a punched cardboard, that is made available in the form of a flat-folded item to be unfolded and erected to provide a urine exhaust conductor to be applied with one larger end thereof around the urinary tract of a standing woman user, thereby providing a urine exhaust duct arranged to direct urine stream falling away from the standing user at its other, usually narrower, end.

U.S. Pat. No. 5,893,176 is directed towards a woman urine device to direct the discharge of urine while standing. The woman urine device is made of plastics and incorporates an inner sealing ring, an outer sealing ring and a funnel. The inner sealing ring is adapted to surround and sealingly engage the exterior of the labia minora. The outer sealing ring lies adjacent the inner sealing ring and is adapted to surround and sealingly engage the exterior of the labia majora. The funnel is located below the inner and outer sealing rings and it is adapted to direct the discharge of urine.

U.S. Pat. No. 5,091,998 is directed towards a funnel device to facilitate urination by women in an upright position. This device includes a semi-rigid funnel rim contoured to surround the female genital region. A flexible funnel body depends from the rim and is sealed to the rim around the circumference. The funnel body has a continuous wall sloped inwardly and towards the front of the funnel body. The funnel body terminates in an orifice situated toward the front of the funnel body. A bendable, elongated disposal tube is sealably attached to the funnel orifice and is inclined downward and outward at an angle with regard to the funnel body. The tube is circumferentially corrugated to enable bending for storage and for directing urine flow.

US 2007/0169253 A1 discloses a disposable funnel to be used by women to direct urine. This funnel comprises a small opening at the bottom of the funnel and a large opening at the top of the funnel.

US 2007/0191795 A1 discloses a more sophisticated disposable urinary device for collecting urinary fluid from a user and directing it into a stream. This device includes an extendible body, a top section and a bottom end section. The top section is coupled to the top of the extendible body around a first opening and has a sidewall that defines a top opening. The bottom end section is coupled below the extendible body around a second opening and has a bottom opening. The extendible body defines an axial passage between the first and second openings and includes a plurality of flexible bellows type folds. The bellows type folds are sufficiently flexible to allow the device to be moveable between an unpackaged position and an extended position. In the unpackaged position, the bottom end section protrudes below the top section to allow a user to grasp the bottom end section. In the extended position, the bellows type folds are extended to form an arc shaped funnel for collecting the urine stream through the top opening and for directing it into a stream that emerges from the bottom opening.

WO 00/15166 describes a disposable funnel-like urinary device comprising a punched sheet of a relatively flexible, although stiff to such an extent as to keep its shape, paper material including: a first and second pair of substantially identical normally flat sections, a relatively small side wing extending along at least a portion of one of said second pair of flat sections and securing means for fixing said side wing to obtain a foldable funnel-like arrangement which in its erected condition constitutes a tubular exhaust duct having a major proximate anatomic urine receiving end designed to be applied around a urinary tract of a standing female user and a smaller distal urine draining end.

With all these known devices, the problem is that they fail to assure liquid-tight connection of the larger end of the known disposable urinary device to the female user's body. Thus, there is still a need for an improved urinary device.

### DISCLOSURE OF THE INVENTION

Hence, it is a general object of the invention to provide a funnel-like urinary device which makes it possible for a woman to urinate in a standing position with good security that the urine is totally collected by and disposed from the funnel-like device.

Another object of the present invention is to provide a funnel-like urinary device which can be stored in a small space, is quite easy and simple to use, preferably is disposable, and much preferred is biologically degradable such that it can be thrown into the toilet. It is also desirable—in particular for a disposable product—that it can be produced at competitive costs.

These and other objects that will appear more clearly below are achieved by a funnel-like urinary device of the present invention as defined in the claims.

Now, in order to implement these and still further objects of the invention, which will become more readily apparent as the description proceeds, the funnel-like urinary device is manifested by the features that it comprises a funnel-like basic body with mirror symmetry formed from a first wall and a second wall, wherein said first wall and said second wall, if pulled apart form a large opening at a first end and a small opening at a second end, wherein the large opening is designed such as to fit the body in the vaginal region such that upon use it is sealed by the labia, in particular or predominantly by the inner labia, and a second end defining a smaller opening for discharging urine upon use and two holding means symmetrically positioned at the outside of the first wall and the second wall, said holding means enabling the user to press the rim of the large opening to the user's body in the vaginal region and covering the exit of the urethra and to open the large opening to such an extent that its outside is in sealing contact with the labia, in particular with the inner labia (labia minora).

In a preferred embodiment, the device is disposable. Disposable in the scope of the present invention means that the device is intended for one single use. Thus, if disposable, the device may be made of a non cleanable material and the material and production costs should be low.

In another preferred embodiment the holding means are tube like bodies designed and positioned such as to receive upon use one finger each, preferably the index and the middle finger, applied from the user's front side, in view of foldability preferably tube like bodies with essentially square cross-section.

The holding means in general are positioned close to the large opening but distant from the rim of said large opening for at least an average size of the labia.

For more comfort and to reduce the risk for injuries, the rim of the large opening is strengthened and rounded, e.g. by being a folded edge or a rolled up edge and not a cut edge.

While the funnel-like urinary device can be made of any sufficiently stiff material that ensures the sealing upon application of slight pressure to the vaginal part of the user's body, for a disposable and foldable device, it is preferred to have a light-weight, cheap material, most preferred a biodegradable material, in particular a biodegradable paper.

Alternatively, in particular for a device that can be used more than once, a material that after use can be washed with water, preferably a water or aqueous liquid resistant and/or repellent material, e.g. plastics or plastic coated paper or impregnated paper, is preferred.

In another preferred embodiment the material may be selected such that it has reduced risk to induce allergies.

Dependent on the material chosen and the number of devices used in the same place, e.g. a public lavatory, collecting and recycling of the devices may be considered.

In a further embodiment, the disposable funnel-like urinary device comprises a cleaning towel attached to the side of the device that upon use is the rear side. With this towel, the user can clean and dry the body part around the exit of the urethra.

For easy and hygienic transport, the disposable funnel-like urinary device of the present invention is folded and packed into a protective bag, preferably a sealed protective bag.

Such bag may be made of paper, plastics, a compound of paper and plastics etc. and sealed by gluing, embossing or by welding dependent on the material used.

For selling, but also for transport, a container containing more than one of the disposable funnel-like urinary devices can be provided. Such a container, usually made of cardboard or impregnated cardboard, may be adapted to house several individually sealed devices or the container may house several “open” devices and being itself closable such that the necessary hygiene of the environment also after a first opening of the container is conserved. A large container may also contain several smaller containers or individually sealed devices, e.g. for use in an automated dispenser that can e.g. be located in a public toilet, lavatory or washroom.

A disposable funnel-like urinary device of the present invention can be produced by preparing a punched first sheet that upon folding provides the funnel-like body, and two punched second sheets that upon folding provide the holding means. The first and second sheets may be of the same or different material(s), preferably the same material which is relatively flexible but sufficiently stiff to regain its shape after a mild deformation, e.g. paper. Then the device is prepared by first joining together the first sheet with the two folded second sheets and finally folding the punched first sheet and joining together two end portions of the sheet, preferably by gluing, although, dependent on the material chosen other methods are usable such as welding, embossing or seaming or stitching, provided that the seam is sufficiently tight.

The punched first sheet has a pair of substantially identical flat essentially V-shaped basic sections that are united along an intermediate longitudinal fold line, which (in use) constitutes the front line of the urinary device. Each section further comprises a top line, a bottom line and a rear line, whereby at least one of the basic sections comprises an ear or flap extending essentially along the whole rear line and serving as basis for a sealing adhesive line connecting the rear lines of the two basic sections thereby forming a funnel-like body. In a less preferred embodiment, the connection of the rear lines may be made by an adhesive tape. It is also possible to connect the rear lines by mere embossment. This has the advantage that no adhesive has to be used. The top line preferably is at least partially curved to improve the fitting of the large opening of the funnel-like device to the vaginal body part of a woman upon use.

The basic sections may be provided with additional ears or flaps that will be folded to provide the preferred folding edge at the rim of the large opening and to also serve to stabilize the upper region of the funnel-like body.

In an alternative embodiment, the upper edge or the rim of the large opening, respectively, is not a folding edge but a rolled up or reeled rim.

In a last step, if desired, a preferably pre-folded cleaning towel may be attached, e.g. by means of an adhesive or by embossment, adjacent to the rear line of the funnel like basic body.

The inventive device is especially suited for use in any public washrooms such as in restaurants, in railway stations, at airports, in trains, in air-crafts and busses, in the theatre, in cinemas, at the working place, for sports and travelling, but also at home if the woman has problems to sit down or to get up again.

A general form as further described herein is suitable for most women from an age of about ten years.

Dependent on the material, the device can be washed and reused or thrown into the toilet or the dustbin.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows a preferred inventive funnel-like urinary device.

FIG. 2 shows a preferred inventive funnel-like urinary device comprising a cleaning towel.

FIG. 3 shows the production steps for producing a preferred device of the present invention with

FIG. 3A showing the punched first sheet with preferred locations of ears and/or flaps.

FIG. 3B shows the first sheet with the ears to and flaps folded into a position that upon final folding will be inside the formed funnel-like body.

FIG. 3C shows one of the punched second sheets

FIG. 3D shows the second sheet after folding the holding means and gluing the flap parts together to form a hollow tube with essentially square cross-section and attaching flaps.

FIG. 3E shows the holding means with one part of the attaching flaps attached (glued) to the inner side of the basic sections.

FIG. 3F shows the holding means with another part of the attaching flaps attached (glued) to the outer side of the basic sections.

FIG. 3G shows the finally folded funnel-like urinary device with the rear line glued and sealed by means of a flap positioned along one of the rear lines.

FIG. 4 shows different steps in the production and attachment of a cleaning towel, wherein

FIG. 4A shows the punched unfolded towel with two attachment straps at one end and lateral folding lines for the towel itself indicated.

FIG. 4B shows the towel in laterally folded position with folding of the straps indicated.

FIG. 4C shows the towel in partially folded form with the last longitudinal folding line indicated.

FIG. 4D is a top view onto the top of the funnel-like urinary device with the cleaning towel attached to the top walls of the two holding means.

FIG. 4E is a top view on the side of the funnel-like urinary device with indication of the last folding step of the cleaning towel.

FIG. 4F shows a preferred funnel-like urinary device in finally folded state.

FIG. 5 shows a preferred sealing package, wherein

FIG. 5A shows the punched material with folding and weakening lines indicated

FIG. 5B shows the folded and sealed final bag with the weakening line being formed and positioned is such that the bag can easily be opened, the funnel-like device grasped close to the large opening and withdrawn from the bag.

FIG. 6 shows the funnel-like device in totally collapsed state, i.e. with the two sides touching each other indicating preferred dimensions in millimeters

FIG. 7 is a top view on the opened funnel-like device, wherein

FIG. 7A shows the usual opening and the funnel-like device which is around 15 mm and

FIG. 7B shows the maximal opening of the funnel like device which is around 40 mm or an opening angle of 65°

FIG. 8 shows the dimensions of a preferred finger lash optimized for a funnel-like device as shown in FIG. 6.

FIG. 9 shows a preferred form of a cleaning towel with dimensions optimized for a funnel-like device with funnel-dimensions as indicated in FIG. 6.

### LEGEND

1 funnel-like urinary device
2 funnel-like body
3 first wall or basic section
4 second wall or basic section
5 large opening
6 small opening
7,8 holding means
9 first sheet
10 longitudinal fold line, front line
11 top line
12 bottom line
13 rear line
14 ear or flap extending essentially along the whole rear line
15 front part ears or flaps
16 rear part ears or flaps
17, 18 second sheets
19 cleaning towel
20, 21 straps
22 lateral folding lines
23 longitudinal folding line
24 strap folding lines
30 sealed protective bag
31 folding line
32 weakening line
40 adhesive comprising or optionally embossed parts, further designated as A to G
71 top wall
72 first side wall
73 bottom wall
74 second side wall
75-77 ears or flaps
78 user assisting, printed mark

### MODES FOR CARRYING OUT THE INVENTION

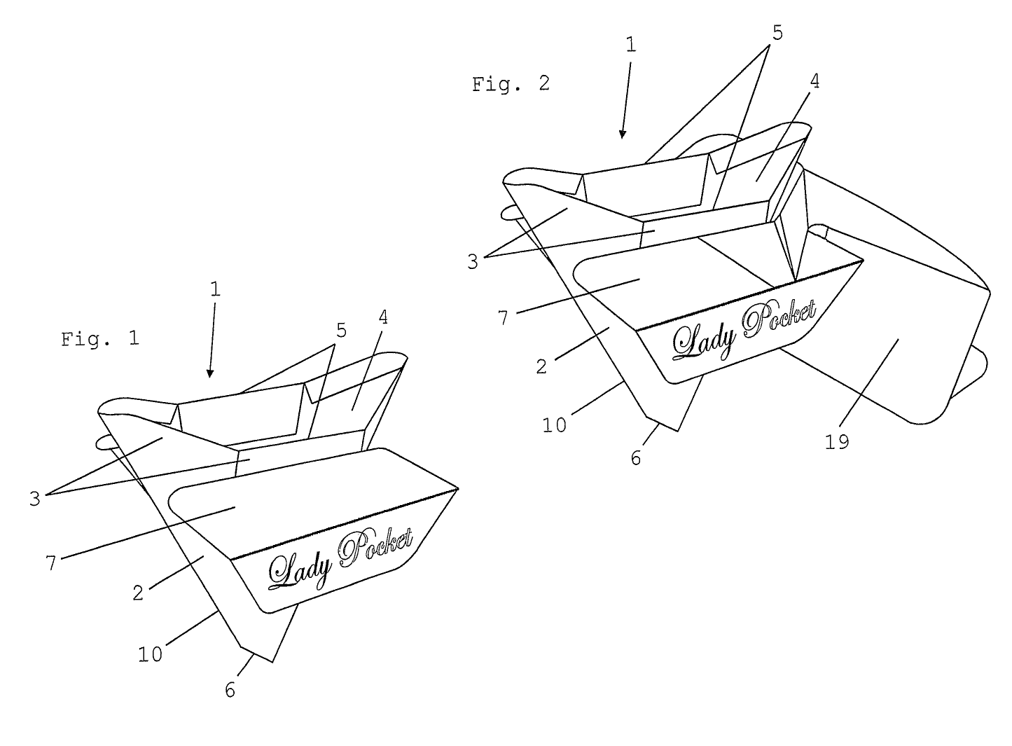

The funnel-like urinary device 1 of the present invention (see FIG. 1) comprises a funnel-like basic body 2 with mirror symmetry formed from a first wall or a first basic section 3 and a second wall or a second basic section 4, wherein said first wall 3 and said second wall 4, if pulled apart form a large opening 5 at a first end and a small opening 6 at a second end, wherein the large opening 5 is designed such as to fit the body in the vaginal region such that upon use it extends over the exit of the urethra and is sealed by the labia, predominantly the inner labia, and a second end defining a smaller opening 6 for discharging urine upon use and two is holding means 7, 8 symmetrically positioned at the outside of the first wall 3 and the second wall 4, said holding means 7, 8 enabling the user to press the rim of the large opening 5 to the user's body in the vaginal region and extending over the exit of the urethra and to slightly open the large opening such that its outside is in sealing contact with the inner labia.

The holding means 7, 8 (see FIGS. 1 and 2) in general are tube like bodies designed and positioned such as to receive upon use one finger each, preferably the index and the middle finger, applied from the user's front side. For foldability, the tube like body has an essentially square cross-section. In addition, in order to better protect the fingers, the top wall 71 of the holding means and the first (outer) sidewall 72 of the holding means distant from the body may be larger such as to cover almost the whole finger.

The holding means 7, 8 are positioned in the upper part of the funnel-like body 2, preferably close to the large opening 5 but distant from the rim of said large opening 5 for at least an average size of the labia. Such distance is e.g. 5 to 9 mm, preferably about 6 mm.

For improved security with regard to improved stability as well as reduced risk of injury, the rim of the large opening is strengthened and rounded, e.g. by being a folded edge or a rolled up edge and not a cut edge. This can be achieved by providing ears or flaps 15, 16 that can be folded into the funnel-like body 2 or by attaching folded parts, such as an ear or flap 74-77 simultaneously used for securing the holding means 7, 8 to the funnel-like body 2.

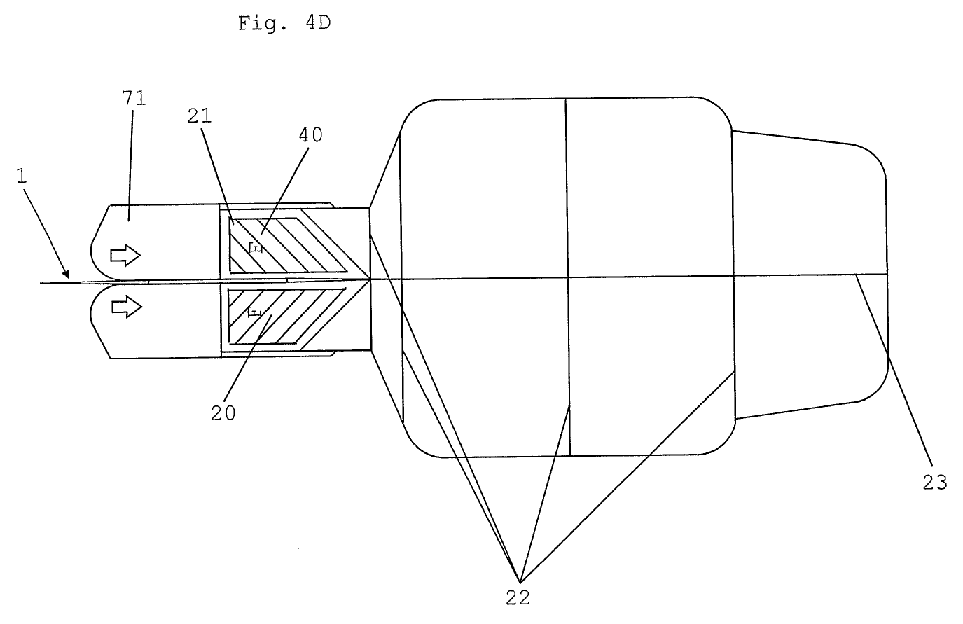

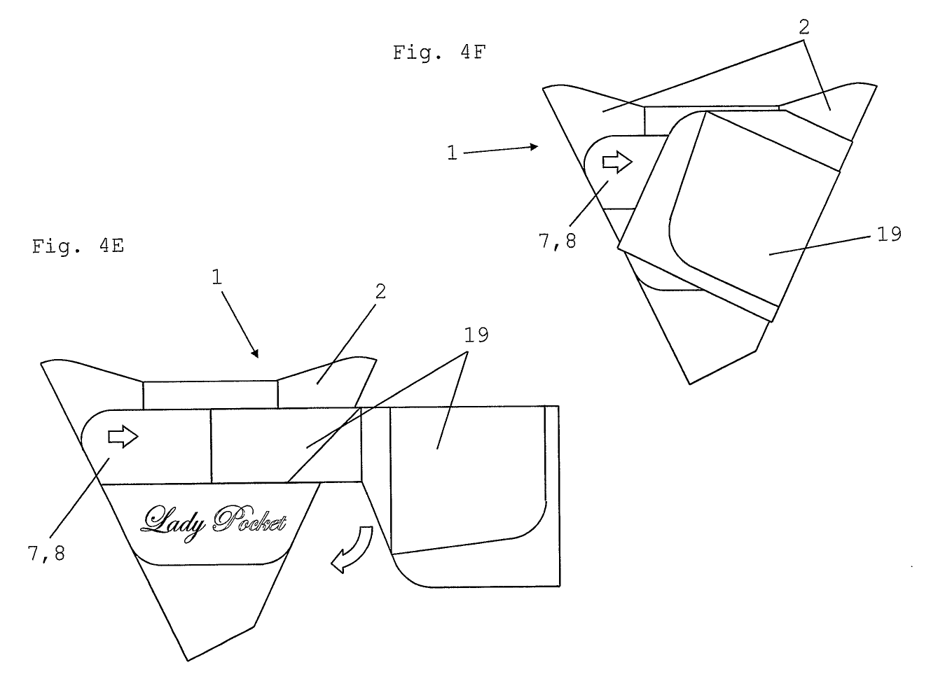

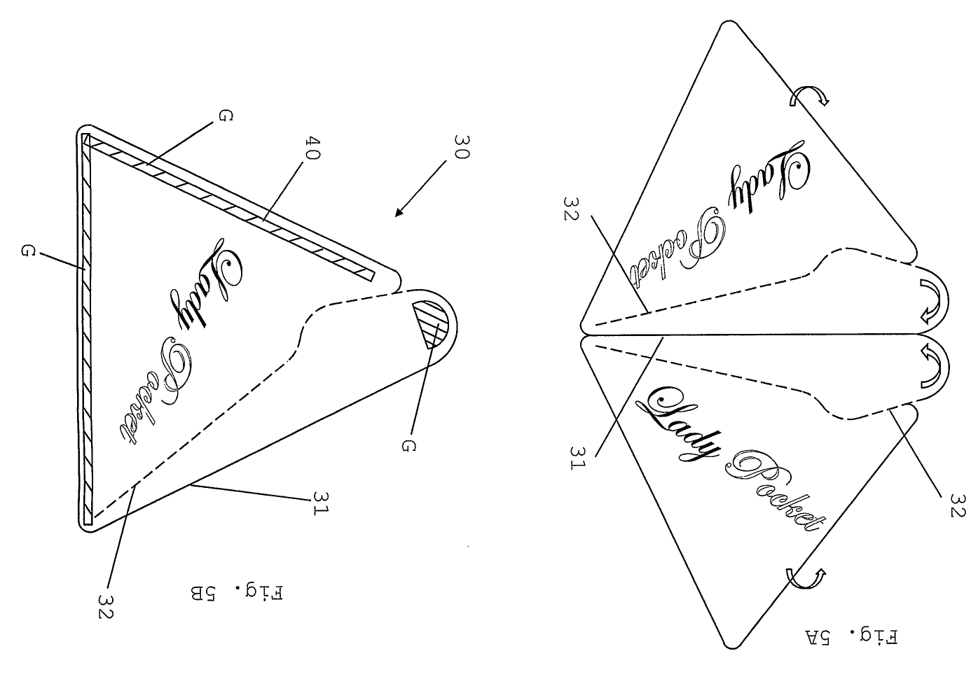

In a preferred embodiment, the rim of the large opening 5 of the funnel-like urinary device 1, also referred to as the rim only, in once folded state wherein the first wall 3 and the second wall 4 touch each other is not linear but has a linear central part that merges on both sides in an obtuse angle into linear side parts that end in rounded parts (see FIGS. 4E and 4F). Preferably, the device has the following approximate dimensions:

| | |
|---|---|
| front line | 74 mm ± 5% |
| rear line | 83 mm ± 5% |
| linear central part of the rim | 34 mm ± 5% |
| linear side parts of the rim | 18 mm ± 5% |
| obtuse angles between central | 163° ± 5% |
| part of the rim and the linear | |
| side parts of the rim designnated | |
| as δ in FIG. 6) | |

The rounded ends of the rim connecting the linear side parts of the rim to the front or rear line, respectively, have a radius of 24 mm ±50.

The above indicated basic dimensions result in an angle between the rear line and the edge of the small opening (designated β in FIG. 6) of 90°, for the angle between the front line and the edge of the small opening (designated γ in FIG. 6) of 141° and for the angle between the rear line or the front line and the linear side parts (designated α in FIG. 6) of 47°.

Further dimensions of a presently preferred device are indicated in FIG. 6, wherein R designates the radius.

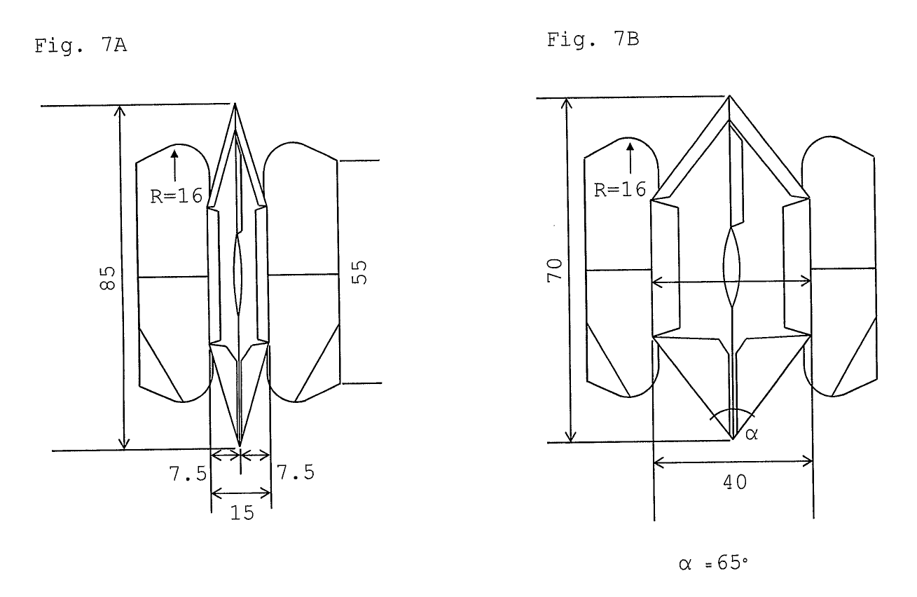

These dimensions that may be readily varied within ±5%, represent presently preferred dimensions, optimized for an average user. Further deviations may be allowed without making the device unusable or too large for easy transport, however, the above indicated dimensions have proved to be suitable to fit the vaginal part of an averagely sized woman, and result in a funnel-like body 2 with a small opening 6 dimensioned such that the urine of an averagely urinating woman can be stored and discharged without “overflow”. These dimensions have been found suitable for a usual opening of the large opening of 15 mm (see FIG. 7A) and they are more than sufficient for an extreme opening of about 40 mm as shown in FIG. 7B.

Alterations that might be made without destroying the usability comprise e.g. a larger front line while keeping the rear line and the rear linear side parts constant, i.e. a slightly reduced angle between the central part of the rim and the linear front side part of the rim (designated δ in FIG. 6). An alternative possibility is to reduce the angle between the rim and the rear line (designated α in FIG. 6) such that the small opening of the funnel is “advanced”. Important with all these amendments is that the volume of the funnel and the dimensions of the small opening are not changed for more than at most about 15%, preferably not more than 10%, much preferred not more than 5%. For good “positioning” of the urine stream, a larger funnel has proved to be less suitable than a funnel with the above indicated dimensions.

It is also possible to prepare such devices for larger or smaller persons by keeping the relations between the dimensions constant.

For a funnel-like device with the dimensions shown above, finger lashes with the following main dimensions are preferred:

| | |
|---|---|
| top wall 71 | 18 mm ± 5% |
| first side wall 72 | 22 mm ± 5% |
| bottom wall 73 | 18 mm ± 5% |
| second side wall 74 | 22 mm ± 5% |
| ear or flap 75 | 12 mm ± 5% |
| ear or flap 76 | 6 mm ± 5% |
| ear or flap 77 | 5.5 mm ± 5%. |

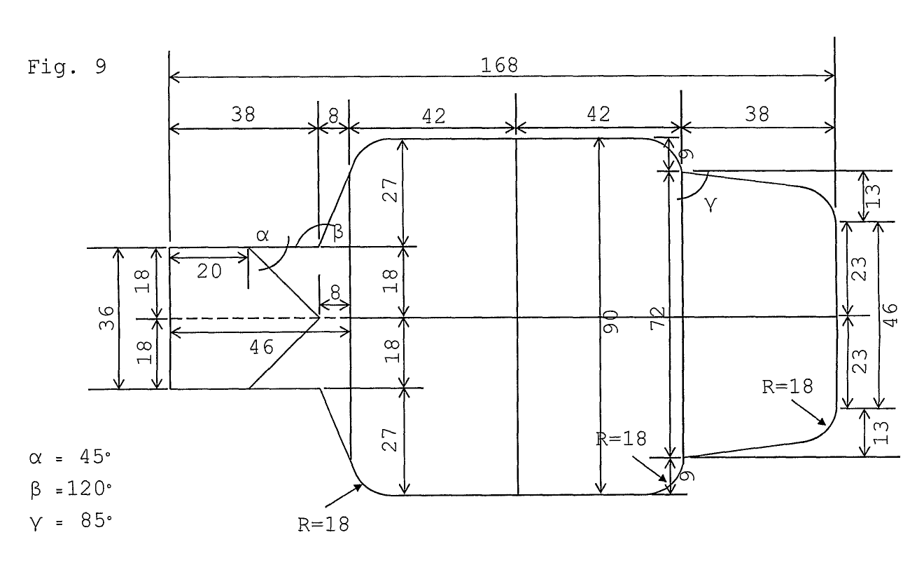

Further dimensions of a presently preferred device are indicated in FIG. 8, wherein R designates the radius.

While the funnel-like urinary device can be made of a great variety of materials such as plastics, paper, card board etc., biodegradable material, in particular biodegradable paper, is preferred. Although for a disposable device use of one kind of biodegradable paper is preferred at present in order to ensure homogeneous biodegradation, it is within the scope of the present invention to use different papers, e.g. thin paper combined with thicker paper or to combine plastics with paper etc.

The preferably disposable funnel-like urinary device 1 of the present invention may additionally comprise a cleaning towel 19 attached to the side of the device that upon use is the rear side (see FIGS. 2 and 4A to 4F).

For optimal fitting to the funnel-like body shown in FIG. 6, such cleaning towel 19 has a longitudinal symmetry axis that corresponds to the longitudinal folding line 23, a total length measured on the symmetry axis of 168 mm and a length of the towel at the symmetry axis projecting over the funnel-like body of 130 mm. This part projecting over the funnel-like body has a central towel body with rounded corners that has also a perpendicular symmetry axis (along the middle perpendicular folding line 22), a longitudinal extension of (2×42 mm) or 84 mm, and a perpendicular extension of (2×45 mm) or 90 mm and a rear part extending from the central part with a length of 38 mm, a dimension perpendicular to the symmetry axis at the connection to the central part of (2×36 mm) or 72 mm and a dimension perpendicular to the symmetry axis at the end of the towel of (2×23 mm) or 46 mm. The front part of the towel comprising the part extending along the funnel-like body extends into two fixing parts with a dimension perpendicular to the symmetry axis of 18 mm each and a length of 46 mm.

All corners of the towel are rounded with a radius of 18 mm.

Further dimensions of a presently preferred towel are indicated in FIG. 9, wherein R designates the radius.

Such a towel allows a folding that totally matches the funnel-like body (see FIG. 4F). Thus, when—the dimensions of the funnel-like body are slightly varied, slight changes to the towel dimensions (in general variations of ±5%) are possible or even recommended.

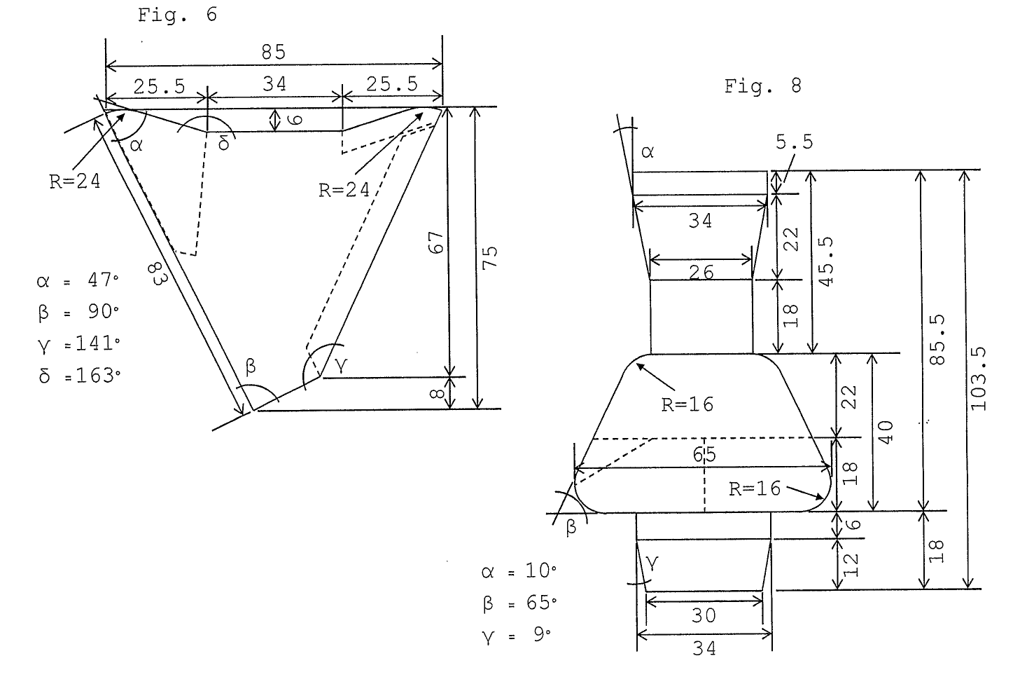

Since the preferred disposable funnel-like urinary device is primarily intended for being used in public toilets, although it can also be used by persons with problems to sit down or stand up due to health problems, age etc. it is preferably folded and packed in a hygienic package, e.g. a protective bag 30, preferably a sealed protective bag 30 (see FIG. 5A for the not yet folded and sealed and FIG. 5B for the folded and sealed bag).

FIG. 5A shows the punched material for forming bag 30 with folding lines 31 and weakening lines 32, e.g. a perforation, indicated. FIG. 5B shows the folded and sealed final bag 30 with the weakening line 32 being formed and positioned such that the bag can easily be opened, the funnel-like device grasped close to the large opening 5 and withdrawn from the bag 30.

The above indicated dimensions for a urinary device 1 allow that it can be stored and protected by a triangular covering bag of about 10 cm length of each of the legs. The material used for such a bag can be a paper, an impregnated paper, plastics or a compound of e.g. paper and plastics etc.

In folded form, in general, the funnel like body is only collapsed such that the two walls touch each other but not further folded. The holding means are folded down such that the top wall 71 and the first, outer side wall 72 become parallel to the bottom wall 73 and the second side wall 74 or the funnel walls 3, 4, respectively. A possibly present towel is preferably symmetrically folded such as to closely fit the two funnel walls 3, 4 (see FIGS. 4E and in particular 4F).

A container containing more than one of the disposable funnel-like urinary devices, preferably about 5 can also be provided. Such container may comprise individually or together packed devices, whereby dependent on whether the devices are individually packed or not a container that can be more or less tightly closed again after taking off one of the devices 1 may be envisaged.

The devices 1 can be sold in stores or provided in public toilets and washrooms, e.g. in dispensers as known for other sanitary goods.

The disposable funnel-like urinary device can be partially hand made (e.g. automatically punched and then the parts folded and connected by hand) or it can be produced fully automated using known, suitably adapted packaging machines, e.g. machines as they are used for individually packaging sanitary napkins or towels, tampons, but also machines for packaging cigarettes, etc.

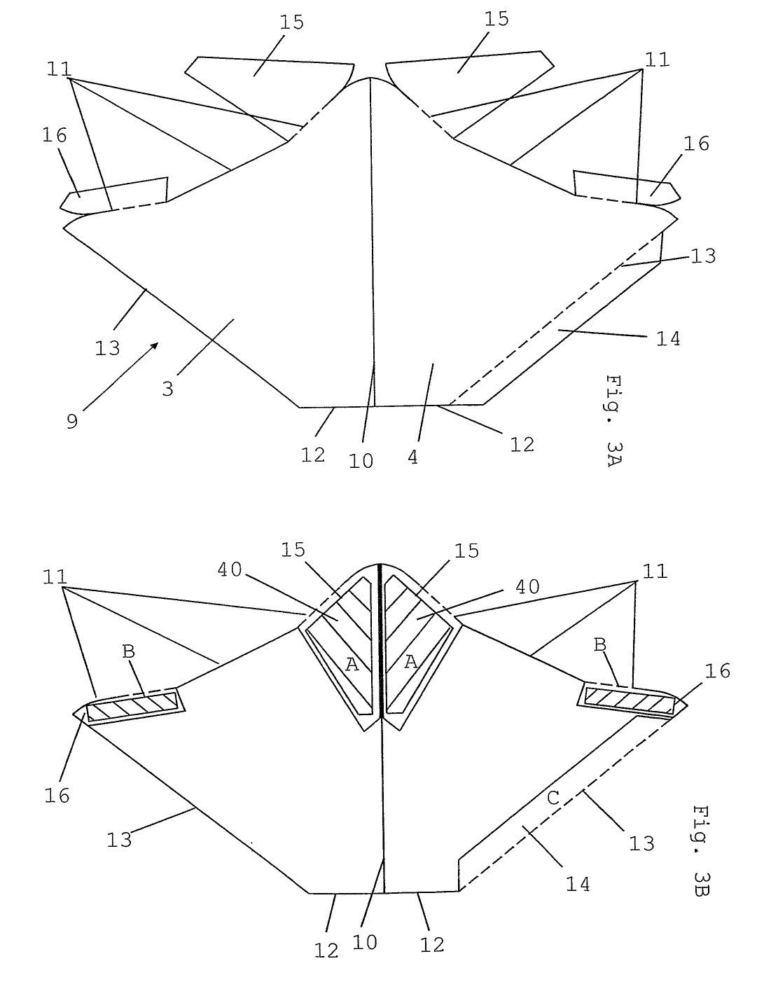

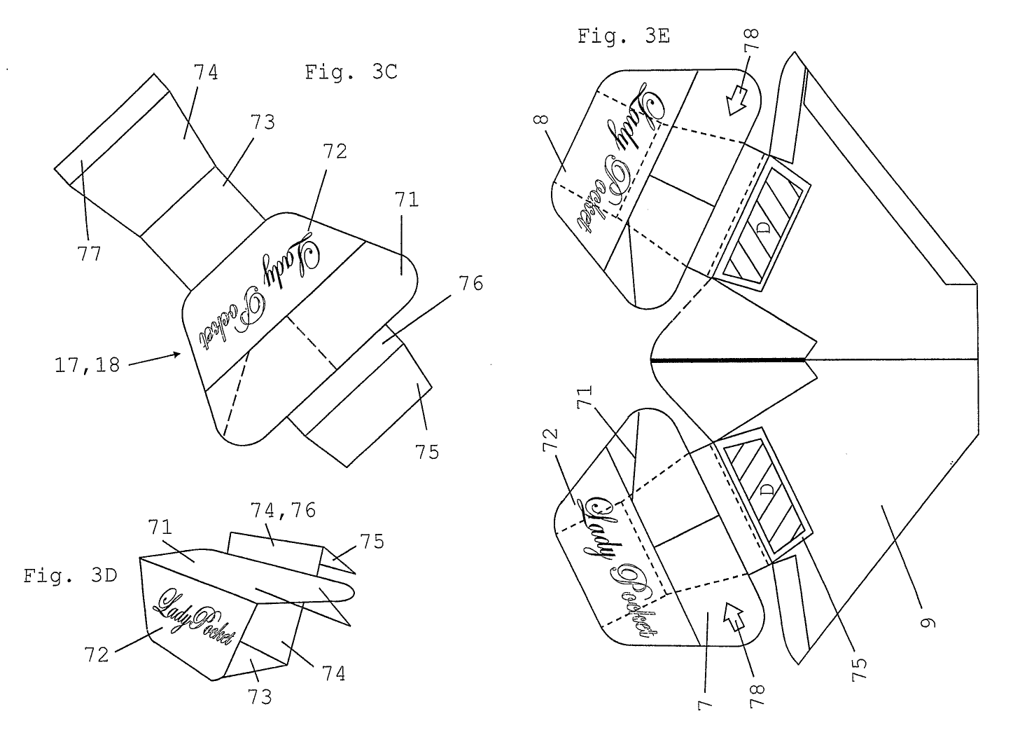

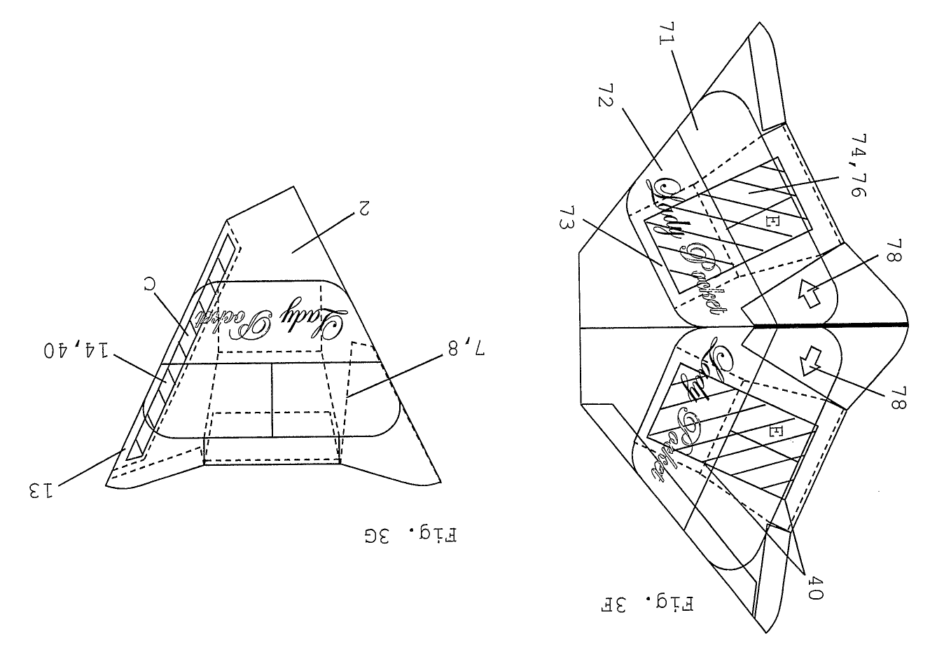

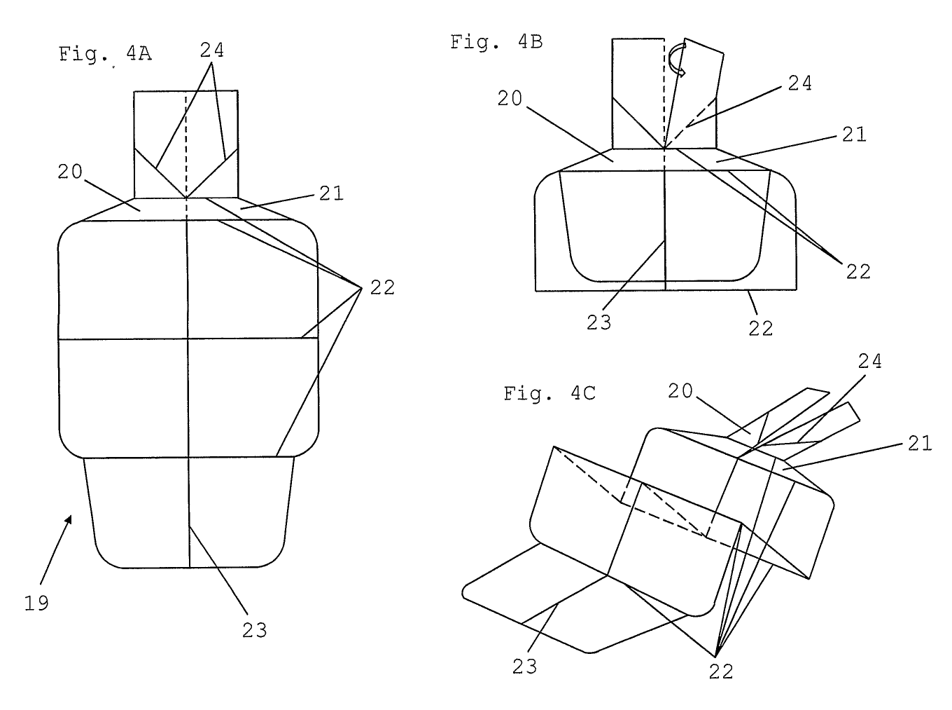

A disposable funnel-like urinary device 1 of the present invention can be produced by preparing a punched first sheet 9 (see FIG. 3A) that upon folding provides the funnel-like body 2 (see FIG. 3G), and two punched second sheets 17, 18 (FIG. 3C) that upon folding provide the holding means 7, 8 (since the holding means 7, 8 are identical or mirror images of one another, only one means, means 7, is further on described in more detail and shown in the Figures, e.g. FIG. 3E). The first and second sheets may be of the same or different material(s), preferably the same material which is relatively flexible but sufficiently stiff to regain its shape after a mild deformation, e.g. paper. Then the device is prepared by first joining together the first sheet 9 with the two folded second sheets 17, 18 (FIGS. 3E and 3F) and finally folding the punched first sheet 9 and joining together two end portions, the rear lines 13 of the sheet, preferably by gluing a flap 14 provided along one of the rear lines 13, e.g. the rear line of basic section or wall 4 to the inner side of the other basic section or wall 3. Although gluing is preferred, dependent on the material chosen other methods are usable such as welding, embossment or seaming or stitching, provided that the necessary tightness of the funnel-like body is conserved.

The punched first sheet (FIG. 3A) has a pair of substantially identical flat essentially V-shaped basic sections that correspond to the first and second walls 3, 4 that are united along an intermediate first longitudinal folding line 10, which (in use) constitutes the front line 10 of the urinary device 1. Each section has a top line 11, a bottom line 12 and a rear line 13, whereby in a preferred embodiment at least one of the basic sections 3, 4 comprises an ear or flap 14 extending essentially along the whole rear line 13 and serving as basis for a sealing adhesive line connecting the rear lines 13 of the two basic sections 3, 4 thereby forming a funnel-like body 2. The top line 11 preferably is at least partially curved to improve the fitting of the large opening of the funnel-like device to the vaginal body part of a woman upon use.

The basic sections 3, 4 may be provided with additional ears or flaps, in particular in areas close to the front line 10 (front part ears or flaps 15) and close to the rear lines 13 (rear part ears or flaps 16) (see FIG. 3A) that will be folded and glued to the side of the basic section that will—after the rear lines 13 have been joined—be the inner side of the funnel-like body 2 (see FIG. 3B). The folded ears on the one hand provide the preferred folded edge at the top line 11 or the large opening 5, respectively, and also serve to stabilize the upper region of the funnel-like body 2. Further ears or flaps may be provided at the top line 11 of the basic sections to ensure that most or all of the rim of the large opening 5 is formed by folded edges. Alternatively and presently preferred, however, the folded edge of the central parts of the top line 11 of each basic section is formed by a folded edge of the second sheets 17, 18 as further described below.

The second sheets 17, 18 may have 6 to 7 parts (see FIG. 3C) that may be folded and connected, e.g. glued together, as shown in FIG. 3D and described below.

The second sheet 17, 18 has parts for providing the top wall 71, the first side wall 72, and the bottom wall 73 of the holding means. The second side wall 74 in folded state extends over the top wall 71 up to the rim of the large opening and optionally extends there via a folded edge into an optional ear or flap 77. For further stability, the tubular part of the holding means 7, 8 is formed by gluing ear or flap 76 to side wall 74 and by optionally also gluing ear or flap 75 to ear or flap 77. This provides further thickness and thus stability. Side wall 74 partially attached to ear or flap 76 is then glued to the outer side of the basic section and the ear or flap 75, optionally glued to ear or flap 77, is glued to the inner side of the same basic section.

In a last step, if desired, a preferably pre-folded cleaning towel 19 may be attached, e.g. by an adhesive 40, adjacent to the rear line of the funnel like basic body (see FIG. 2).

The folding of the cleaning towel 19 is shown in FIG. 4. First a cleaning towel 19 is provided by punching, said cleaning towel 19 has two straps 20 and 21 used for attaching the towel 19 to the top wall 71 of the holding means 7. The reduced diameter of the towel in the part opposite the straps is merely due to the fitting to the funnel-like body in folded state without a further folding step. Lateral folding lines 22, a longitudinal folding line 23 and strap folding lines 24 suitable for such a towel are indicated in FIGS. 4A, 4B and 4C. FIG. 4D shows the folded and attached towel, with adhesive 40 comprising parts indicated.

For gluing the funnel-like body, the ears or flaps, the holding means and/or the cleaning towel, any adhesive may be used, whereby—due to the preferred use of a biodegradable material—a non-toxic, preferably also biodegradable adhesive is preferred.

Alternatively to gluing, connection by special embossment can be used, e.g. for paper products etc.

For use, the user may introduce two fingers into the holding means as indicated by the arrow in e.g. FIG. 3E. Then she may press the upper rim towards her body in the vaginal region and slightly open the large opening 5. In general, the two walls 3,4 will be separated for about 15 mm (see FIG. 7A), although—dependent on the age of the user—opening up to 40 mm is possible (see FIG. 7B).

While there are shown and described presently preferred embodiments of the invention, it is to be distinctly understood that the invention is not limited thereto but may be otherwise variously embodied and practiced within the scope of the following claims.

1. A funnel-like urinary device comprising a funnel-like basic body with mirror symmetry formed from a first wall and a second wall, wherein said first wall and said second wall, if pulled apart, form a large opening at a first end and a small opening at a second end, wherein the large opening is designed such as to fit a woman's body in the vaginal region such that upon use it is sealed by the labia, and a second end defining a smaller opening for discharging urine upon use and two holding means symmetrically positioned at the outside of the first wall and the second wall, said holding means enabling the user to press the rim of the large opening to the user's body in the vaginal region and to slightly open the large opening such that its outside is in sealing contact with the labia.

2. The funnel-like urinary device of claim 1, wherein the holding means are tube like bodies designed and positioned such as to receive upon use one finger each, preferably the index and the middle finger, applied from the user's front side.

3. The funnel-like urinary device of claim 2, wherein the tube like body has an essentially square cross-section.

4. The funnel-like urinary device of claim 2, wherein the holding means are positioned close to the large opening but distant from the rim of said first opening for at least an average size of the labia.

5. The funnel-like urinary device of claim 1, wherein the rim of the large opening is strengthened and rounded, e.g. by being a folded edge and not a cutting edge.

6. The funnel-like urinary device of claim 1, wherein the rim of the large opening in once folded state wherein the first wall and the second wall touch each other is not linear but has a linear central part that merges on both sides in an obtuse angle into linear side parts that end in rounded parts.

7. The funnel-like urinary device of claim 6, having approximately the following dimensions:

| | |
|---|---|
| front line | 74 mm ± 5% |
| rear line | 83 mm ± 5% |
| linear central part of the rim | 34 mm ± 5% |
| linear side parts of the rim | 18 mm ± 5% |
| obtuse angles between central part of | 163° ± 5% |
| the rim and the linear side parts of | (radius 24 mm ± 5%) |
| the rim rounded ends of the rim | |

connecting the linear side parts of the rim to the front or rear line, respectively.

8. The funnel-like urinary device of claim 1, which is disposable.

9. The funnel-like urinary device of claim 8 which is made of biodegradable material, in particular biodegradable paper.

10. The funnel-like urinary device of claim 1, which comprises a cleaning towel attached to the side of the device that upon use is the rear side.

11. The funnel-like urinary device of claim 1, which is folded and packed into a protective bag, preferably a sealed protective bag.

12. A container containing more than one of the funnel-like urinary devices of claim 1.

13. A method for producing a funnel-like urinary device of claim 1, comprising the steps of
preparing a punched first sheet that upon folding provides the funnel-like body,
preparing two punched second sheets that upon folding provide the holding means,
folding the second sheets to provide holding means,
joining together the first sheet with the two folded second sheets or holding means, respectively, and then
folding the punched first sheet and sealingly joining together the rear lines of the sheet, preferably by gluing a flap provided along one of the rear lines to the inner side of the other basic section or wall.

14. The method of claim 13, wherein a pre-folded cleaning towel is attached to the holding means such that in unfolded form it extends beyond the rear side of the funnel like basic body.
